(19) United States
(12) Patent Application Publication
(10) Pub. No.: US 2009/0012053 A1
(43) Pub. Date: Jan. 8, 2009

(54) Use of LXR agonists for the treatment of osteoarthritis

(75) Inventors: Sunil Nagpal, Collegeville, PA (US); Zhiyong Yang, Newtown, MA (US); Elisabeth Morris, Sherborn, MA (US); Edward R. LaVallie, Harvard, MA (US); Lisa A. Collins-Racie, Acton, MA (US)

Correspondence Address: POTTER ANDERSON & CORROON LLP/WYETH, 1313 NORTH MARKET STREET, HERCULES PLAZA, SIXTH FLOOR, WILMINGTON, DE, 19801, US

(73) Assignee: Wyeth, Madison, NJ (US)

(21) Appl. No.: 11/901,513

(22) Filed: Sep. 18, 2007

Related U.S. Application Data
(60) Provisional application No. 60/845,576, filed on Sep. 19, 2006.

(51) Int. Cl. A61K 31/575; A61K 31/18; A61K 31/195; A61K 31/506; A61K 31/42; A61P 19/02; C12Q 1/68

(52) U.S. Cl. 514/182; 514/602; 514/561; 514/269; 514/378; 435/6

(57) ABSTRACT

Disclosed herein are methods of preventing and treating osteoarthritis through the use of LXR agonists.

### CROSS-REFERENCE TO RELATED APPLICATION

This application claims priority from U.S. Provisional Application No. 60/845,576 filed Sep. 19, 2006, which is incorporated by reference herein in its entirety.

### FIELD OF THE INVENTION

The present invention relates to methods of treating or preventing osteoarthritis with LXR agonists.

### BACKGROUND OF THE INVENTION

Osteoarthritis, also known as degenerative joint disease, is characterized by degeneration of articular cartilage as well as proliferation and remodeling of subchondral bone. The usual symptoms are stiffness, limitation of motion, and pain. Osteoarthritis is the most common form of arthritis, and prevalence rates increase markedly with age.

Existing osteoarthritis treatment approaches include exercise, medicines, rest and joint care, surgery, pain relief techniques, alternative therapies, and weight control. The commonly used medicines in treating osteoarthritis include nonsteroidal anti-inflammatory drugs (NSAIDs), for example, aspirin, ibuprofen, naproxen sodium, ketoprofen; topical pain-relieving creams, rubs, and sprays (for example, capsaicin cream) applied directly to the skin; corticosteroids, typically injected into affected joints to relieve pain temporarily; and hyaluronic acid. Surgery may be performed to resurface (smooth out) bones, reposition bones, and replace joints. Although various medications have been used for treating the disease, they are not effective for long term control and prevention.

Liver X receptors (LXRs), originally identified from liver as orphan receptors, are members of the nuclear hormone receptor super family and have been found to be negative regulators of macrophage inflammatory gene expression (see Published U.S. Patent Application No. 2004/0259948; Joseph S B et al., Nat. Med. 9:213-19 (2003)). LXRs are ligand-activated transcription factors and bind to DNA as obligate heterodimers with retinoid X receptors. While LXRα is restricted to certain tissues such as liver, kidney, adipose, intestine, and macrophages, LXRβ displays a ubiquitous tissue distribution pattern. Activation of LXRs by oxysterols (endogenous ligands) in macrophages results in the expression of several genes involved in lipid metabolism and reverse cholesterol transport, including ABCA1, ABCG1, and apolipoprotein E.

### SUMMARY OF THE INVENTION

One aspect is for a method for the treatment of a mammal suffering from osteoarthritis comprising administering to the mammal in need thereof an LXR-responsive gene expression-inducing amount of an LXR agonist.

Another aspect is for a method of inducing expression of apolipoprotein D in a mammal having osteoarthritic cartilage comprising administering to the mammal in need thereof an effective amount of an LXR agonist.

A further aspect relates to a method of preventing osteoarthritis comprising: (a) determining a baseline apolipoprotein D expression level in normal cartilage of a subject; and (b) maintaining baseline apolipoprotein D expression level in cartilage of the subject via treatment with LXR agonist.

An additional aspect is for a method for the treatment of a mammal suffering from osteoarthritis comprising administering to the mammal in need thereof an aggrecanase activity-inhibiting amount of an LXR agonist.

A further aspect is for a method of inhibiting activity of aggrecanase in a mammal having osteoarthritic cartilage comprising administering to the mammal in need thereof an effective amount of an LXR agonist.

Another aspect relates to a method for the treatment of a mammal suffering from osteoarthritis comprising administering to the mammal in need thereof an effective amount of an LXR agonist to inhibit elaboration of pro-inflammatory cytokines in osteoarthritic lesions.

An additional aspect relates to a method of detecting an osteoarthritic phenotype in a subject comprising: (a) determining a baseline apolipoprotein D expression level in normal cartilage; (b) obtaining a cartilage sample from a subject suspected of having osteoarthritis; and (c) detecting the level of expression of apolipoprotein D in the sample; wherein a lower amount of apolipoprotein D expression in the sample compared to baseline apolipoprotein D expression is indicative of osteoarthritis.

A further aspect is for a method of identifying an LXR ligand capable of reducing an osteoarthritic effect in cartilage comprising: (a) providing a sample containing LXR; (b) contacting the sample with a test compound; and (c) determining whether the test compound induces apolipoprotein D expression, inhibits aggrecanase activity, inhibits elaboration of pro-inflammatory cytokines, or a combination thereof.

Other aspects and advantages of the present invention will become apparent to those skilled in the art upon reference to the detailed description that hereinafter follows.

### BRIEF DESCRIPTION OF THE FIGURES

FIG. 1A is a bar graph showing relative expression levels of nuclear receptor (NR) expression in cartilage with severe osteoarthritis (OA). FIG. 1B is a bar graph showing relative expression levels of retinoid receptor expression in cartilage with severe OA.

FIG. 2A is a bar graph showing ApoD expression in normal cartilage, and cartilage with mild OA and severe OA. Disease severity was assessed macroscopically by examining the sizes and depth of the lesions in the cartilage specimens. FIG. 2B is a bar graph showing TNFα expression in normal cartilage, and cartilage with mild OA and severe OA.

FIG. 3 is a bar graph showing that cytokine-induced proteoglycan degradation/release from human OA cartilage explants is inhibited by LXR agonists, and that cytokine-induced reduction of total proteogycan content in these explants is prevented by LXR agonists.

FIG. 4A is a Western blot showing aggrecanase-generated aggrecan neoepitopes using BC-3 antibody, which recognizes the N-terminus on aggrecanase-generated aggrecan catabolites. Cartilage explants from two human donors with end stage OA (after joint replacement surgery) were used. Donor #259 is a 57 year-old male patient, and donor #261 is a 55 year-old female patient. Lanes 1, 5: vehicle. Lanes 2, 6: TO901317 (2 μM). Lanes 3, 7: IL-1β+oncostatin M (OSM) (10 ng/ml each). Lanes 4, 8: IL-1β+OSM+TO901317.

FIG. 4B is a Western blot showing aggrecanase-generated aggrecan neoepitopes using AGEG antibody, which recognizes a different epitope on aggrecanase-generated aggrecan catabolites. Lanes 1, 5: vehicle. Lanes 2, 6: TO901317 (2 μM). Lanes 3, 7: IL-1β+OSM (10 ng/ml each). Lanes 4, 8: IL-1β+OSM+TO901317.

FIG. 5A is a bar graph showing inhibition of total prostaglandin E2 (PGE2) production from cytokine-treated human cartilage explants by LXR agonists.

FIG. 5B compares the quantities of arachidonic acid in the forms of membrane phospholipids PC and PE in the explants treated with vehicle control or LXR agonist GW3965 (2 μM) for 21 days. Cartilage samples from 2 human OA donors were used in this study.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide to Molecular Cloning (1984); Methods in Enzymology (Academic Press, Inc., N.Y.); Gene Transfer Vectors for Mammalian Cells (J. H. Miller and M. P. Calos eds., 1987, Cold Spring Harbor Laboratory); Methods in Enzymology, Vols. 154 and 155 (Wu et al. eds.), Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Here, Applicants show that LXRα and LXRβ (liver X receptor α and β) are expressed in normal, medium osteoarthritic, and severe osteoarthritic cartilages. Applicants also demonstrate for the first time a plausible lipid defect in osteoarthritis because the expression of Apolipoprotein D (ApoD), which is expressed at a very high level in normal cartilage, is dramatically down regulated in medium and severe osteoarthritic cartilage. LXR ligands induce the expression of ApoD via an LXR responsive element present in the ApoD promoter region. In accordance with the expression data, protein levels of proapolipoprotein D are also reduced in osteoarthritic cartilage samples when compared to normal cartilage. Because ApoD is a lipid (arachidonic acid and cholesterol) binding protein, its reduction in osteoarthritic cartilage may account for increased lipid levels that are observed in the osteoarthritic cartilage. Increased arachidonic acid in the cartilage is expected to result in increased levels of lipid mediators of inflammation (PGE2, leukotrienes, and the like) in the diseased tissue. Osteoarthritic cartilage also shows increased activity of cartilage-degrading enzymes (aggrecanases and metalloproteases).

Applicants also show for the first time that LXR ligand inhibits the activity of aggrecanases in human osteoarthritis articular cartilage tissue explants. LXR ligands also inhibit the expression of TNFα, and a number of other pro-inflammatory cytokines. Therefore, an LXR ligand is expected to be therapeutically efficacious in osteoarthritis, and more efficacious than the current as well as upcoming osteoarthritic therapies, by normalizing the lipid defect, inhibiting the expression and/or activity of aggrecanases/metalloproteases, and inhibiting the elaboration of pro-inflammatory cytokines in osteoarthritic lesions. Further, LXR ligands induce the c-jun/c-fos family of proteins and, as a result, enhance AP1 activity, which is required for cartilage formation. Therefore, with LXR ligands, for the first time, an osteoarthritis treatment may not only inhibit cartilage degradation but also may induce cartilage regeneration.

### I. DEFINITIONS

In the context of this disclosure, a number of terms shall be utilized.

As used herein, the term “about” or “approximately” means within 20%, preferably within 10%, and more preferably within 5% of a given value or range.

The term “aggrecanase activity” refers to at least one cellular process interrupted or initiated by an aggrecanase enzyme binding to aggrecan. Generally, activity refers to proteolytic cleavage of aggrecan by aggrecanase. Other aggrecanase activities include, but are not limited to, binding of aggrecanase to aggrecan and a biological response resulting from the binding to or cleavage of aggrecan by aggrecanases.

The term “cytokine elaboration” refers to production of cytokines by cartilaginous tissue or chondrocytes.

The terms “effective amount”, “therapeutically effective amount”, “an LXR-responsive gene expression-inducing amount”, “aggrecanase activity-inhibiting amount”, and “effective dosage” as used herein, refer to the amount of an effector molecule that, when administered to a mammal in need, is effective to at least partially ameliorate or to at least partially prevent conditions related to osteoarthritis.

As used herein, the term “expression” includes the process by which DNA is transcribed into mRNA and translated into polypeptides or proteins.

The term “induce” or “induction” of apolipoprotein D (ApoD) expression refers to an increase, induction, or otherwise augmentation of apolipoprotein D mRNA and/or protein expression. The increase, induction, or augmentation can be measured by one of the assays provided herein. Induction of apolipoprotein D expression does not necessarily indicate maximal expression of apolipoprotein D. An increase in ApoD expression can be, for example, at least about 10%, 20%, 30%, 40%, 50%, 60%, 70%, 80%, 90% or more. In one embodiment, induction is measured by comparing ApoD mRNA expression levels from normal cartilage to that of ApoD mRNA expression levels from osteoarthritic cartilage.

The term “inhibit” or “inhibition” of aggrecanase or aggrecanase activity refers to a reduction, inhibition, or otherwise diminution of at least one activity of aggrecanase. The reduction, inhibition, or diminution of binding can be measured by one of the assays provided herein. Inhibition of aggrecanase activity does not necessarily indicate a complete negation of aggrecanase activity. A reduction in activity can be, for example, at least about 10%, 20%, 30%, 40%, 50%, 60%, 70%, 80%, 90% or more. In one embodiment, inhibition is measured by a reduction in the detection of cleavage products of aggrecan.

The term “inhibit” or “inhibition” of elaboration of pro-inflammatory cytokines refers to a reduction, inhibition, or otherwise diminution of the activity of a cytokine such as, for example, iNOS, MCP-3, COX-2, MIP1β, MMP-9, IP-10, IL-1β, IL-1α, G-CSF, TNFα, MCP-1, IL-6. The reduction, inhibition, or diminution of cytokine elaboration can be measured by one of the assays provided herein. Inhibition of pro-inflammatory cytokine elaboration does not necessarily indicate a complete negation of pro-inflammatory cytokine elaboration. A reduction in elaboration can be, for example, at least about 10%, 20%, 30%, 40%, 50%, 60%, 70%, 80%, 90% or more. In one embodiment, inhibition is measured by comparing TNFα mRNA expression levels from normal cartilage to that of TNFα mRNA expression levels from osteoarthritic cartilage.

“Liver X receptor” or “LXR” refers to both LXRα and LXRβ, and variants, isoforms, and active fragments thereof. LXRβ is ubiquitously expressed, while LXRα expression is limited to liver, kidney, intestine, spleen, adipose tissue, macrophages, skeletal muscle, and, as demonstrated herein, cartilage. Representative GenBank® accession numbers for LXRα sequences include the following: human (Homo sapiens, Q13133), mouse (Mus musculus, Q9Z0Y9), rat (Rattus norvegicus, Q62685), cow (Bos taurus, Q5E9B6), pig (Sus scrofa, AAY43056), chicken (Gallus gallus, AAM90897). Representative GenBank® accession numbers for LXRβ include the following: human (Homo sapiens, P55055), mouse (Mus musculus, Q60644), rat (Rattus norvegicus, Q62755), cow (Bos taurus, Q5BIS6).

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

The term “modulate” encompasses either a decrease or an increase in activity or expression depending on the target molecule. For example, an ApoD modulator is considered to modulate the expression of ApoD if the presence of such ApoD modulator results in an increase or decrease in ApoD expression.

### II. LXR AGONISTS

LXR agonists useful in the present invention include natural oxysterols, synthetic oxysterols, synthetic nonoxysterols, and natural nonoxysterols. Exemplary natural oxysterols include 20(S) hydroxycholesterol, 22(R) hydroxycholesterol, 24(S) hydroxycholesterol, 25-hydroxycholesterol, 24(S), 25 epoxycholesterol, and 27-hydroxycholesterol. Exemplary synthetic oxysterols include N,N-dimethyl-3β-hydroxycholenamide (DMHCA). Exemplary synthetic nonoxysterols include N-(2,2,2-trifluoroethyl)-N-{4-[2,2,2-trifluoro-1-hydroxy-1-(trifluoromethyl)ethyl]phenyl}benzene sulfonamide (TO901317; Tularik 0901317), [3-(3-(2-chloro-trifluoromethylbenzyl-2,2-diphenylethylamino)propoxy)phenylacetic acid] (GW3965), N-methyl-N-[4-(2,2,2-trifluoro-1-hydroxy-1-trifluoromethyl-1-ethyl)-phenyl]-benzenesulfonamide (TO314407), 4,5-dihydro-1-(3-(3-trifluoromethyl-7-propyl-benzisoxazol-6-yloxy)propyl)-2,6-pyrimidinedione, 3-chloro-4-(3-(7-propyl-3-trifluoromethyl-6-(4,5)-isoxazolyl)propylthio)-phenyl acetic acid (F₃-MethylAA), and acetyl-podocarpic dimer. Exemplary natural nonoxysterols include paxilline, desmosterol, and stigmasterol.

Other useful LXR agonists are disclosed, for example, in Published U.S. Patent Application Nos. 2006/0030612, 2005/0131014, 2005/0036992, 2005/0080111, 2003/0181420, 2003/0086923, 2003/0207898, 2004/0110947, 2004/0087632, 2005/0009837, 2004/0048920, and 2005/0123580; U.S. Pat. Nos. 6,316,503, 6,828,446, 6,822,120, and 6,900,244; WO01/41704; Menke J G et al., Endocrinology 143:2548-58 (2002); Joseph S B et al., Proc. Natl. Acad. Sci. USA 99:7604-09 (2002); Fu X et al., J. Biol. Chem. 276:38378-87 (2001); Schultz J R et al., Genes Dev. 14:2831-38 (2000); Sparrow C P et al., J. Biol. Chem. 277:10021-27 (2002); Yang C et al., J. Biol. Chem., Manuscript M603781200 (Jul. 20, 2006); Bramlett K S et al., J. Pharmacol. Exp. Ther. 307:291-96 (2003); Ondeyka J G et al., J. Antibiot (Tokyo) 58:559-65 (2005).

### III. METHODS OF TREATMENT/PREVENTION

According to one modulatory method, LXR activity is stimulated in a cell by contacting the cell with an LXR agonist. Examples of such LXR agonists are described above in Section II. Other LXR agonists that can be used to stimulate the LXR activity can be identified using screening assays that select for such compounds, as described in detail herein (Section V).

Modulatory methods can be performed in vitro (e.g., by culturing the cell with an LXR agonist or by introducing an LXR agonist into cells in culture) or, alternatively, in vivo (e.g., by administering an LXR agonist to a subject or by introducing an LXR agonist into cells of a subject). For practicing a modulatory method in vitro, cells can be obtained from a subject by standard methods and incubated (i.e., cultured) in vitro with an LXR agonist to modulate LXR activity in the cells.

### 1. Prophylactic Methods

In one aspect, the invention provides a method for preventing in a subject osteoarthritis by administering to the subject an LXR agonist that induces ApoD expression and/or inhibits aggrecanase activity and/or inhibits the elaboration of pro-inflammatory cytokines in osteoarthritic lesions. Administration of a prophylactic LXR agonist can occur prior to the manifestation of osteoarthritis symptoms, such that osteoarthritis is prevented or, alternatively, delayed in its progression.

### 2. Therapeutic Methods

Another aspect of the invention pertains to methods of modulating LXR activity for osteoarthritis therapeutic purposes. Accordingly, in an exemplary embodiment, a modulatory method of the invention involves contacting a cell with an LXR agonist that modulates ApoD expression and/or aggrecanase activity and/or inhibits the elaboration of pro-inflammatory cytokines in osteoarthritic lesions. These modulatory methods can be performed in vitro (e.g., by culturing the cell with an LXR agonist) or, alternatively, in vivo (e.g., by administering an LXR agonist to a subject). As such, the present invention provides methods of treating an individual afflicted with osteoarthritis that would benefit from modulation of ApoD expression and/or aggrecanase activity and/or pro-inflammatory cytokine elaboration in osteoarthritic lesions.

### IV. ADMINISTRATION OF LXR AGONISTS

LXR agonists are administered to subjects in a biologically compatible form suitable for pharmaceutical administration in vivo to enhance ApoD expression and/or suppress aggrecanase activity and/or suppress elaboration of pro-inflammatory cytokines. By “biologically compatible form suitable for administration in vivo” is meant a form of the LXR agonist to be administered in which any toxic effects are outweighed by the therapeutic effects of the agonist. The term “subject” is intended to include living organisms in which an immune response can be elicited, for example, mammals. Administration of LXR agonists as described herein can be in any pharmacological form including a therapeutically effective amount of an LXR agonist alone or in combination with a pharmaceutically acceptable carrier.

A therapeutically effective amount of an LXR agonist may vary according to factors such as the disease state, age, sex, and weight of the individual, and the ability of the LXR agonist to elicit a desired response in the individual. Dosage regime may be adjusted to provide the optimum therapeutic response. For example, several divided doses may be administered daily, or the dose may be proportionally reduced as indicated by the exigencies of the therapeutic situation.

The therapeutic or pharmaceutical compositions of the present invention can be administered by any suitable route known in the art including, for example, oral, intravenous, subcutaneous, intramuscular, transdermal, intrathecal, or intracerebral or administration to cells in ex vivo treatment protocols. Administration can be either rapid as by injection or over a period of time as by slow infusion or administration of slow release formulation. For treating or preventing osteoarthritis, administration of the therapeutic or pharmaceutical compositions of the present invention can be performed, for example, by oral administration or by intra-articular injection.

Furthermore, LXR agonists can be stably linked to a polymer such as polyethylene glycol to obtain desirable properties of solubility, stability, half-life, and other pharmaceutically advantageous properties (see, e.g., Davis et al., Enzyme Eng. 4:169-73 (1978); Burnham N L, Am. J. Hosp. Pharm. 51:210-18 (1994)).

LXR agonists can be in a composition that aids in delivery into the cytosol of a cell. For example, an LXR agonist may be conjugated with a carrier moiety such as a liposome that is capable of delivering the agonist into the cytosol of a cell. Such methods are well known in the art (see, e.g., Amselem S et al., Chem. Phys. Lipids 64:219-37 (1993)). In addition, an LXR agonist can be delivered directly into a cell by microinjection.

LXR agonists can be employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the LXR agonist, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. LXR agonists can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation.

Dose administration can be repeated depending upon the pharmacokinetic parameters of the dosage formulation and the route of administration used.

It is also provided that certain formulations containing LXR agonists are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents, or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the LXR agonist and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the LXR agonist activities disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such LXR agonists can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. LXR agonists that exhibit large therapeutic indices are preferred. While LXR agonists that exhibit toxic side effects may be used, care should be taken to design a delivery system that targets such agonists to the site of affected tissue in order to minimize potential damage to uninfected cells and, thereby, reduce side effects.

The data obtained from the cell culture assays and animal studies can be used in formulating a range of dosage for use in humans. The dosage of such LXR agonists lies preferably within a range of circulating concentrations that include the ED₅₀ with little or no toxicity. The dosage may vary within this range depending upon the dosage form employed and the route of administration utilized. For any LXR agonist used in a method of the invention, the therapeutically effective dose can be estimated initially from cell culture assays. A dose may be formulated in animal models to achieve a circulating plasma concentration range that includes the IC₅₀ (i.e., the concentration of LXR agonist that achieves a half-maximal inhibition of symptoms) as determined in cell culture. Such information can be used to more accurately determine useful doses in humans. Levels in plasma may be measured, for example, by high performance liquid chromatography.

Monitoring the influence of LXR agonists on the expression of ApoD and/or activity of aggrecanase and/or the elaboration of pro-inflammatory cytokines can be applied not only in basic drug screening, but also in clinical trials. For example, the effectiveness of an LXR agonist can be monitored in clinical trials of subjects exhibiting decreased ApoD gene expression in chondrocytes and/or increased aggrecanase activity and/or increased elaboration of pro-inflammatory cytokines in osteoarthritic lesions. In such clinical trials, the expression of ApoD and/or the activity of aggrecanase and/or the elaboration of pro-inflammatory cytokines can be used as a “read out” or markers of the phenotype of different osteoarthritis stages.

Thus, to study the effect of LXR agonists on osteoarthritis, for example, in a clinical trial, cells can be isolated and RNA prepared and analyzed for the levels of expression of ApoD and other genes implicated in osteoarthritis (for example, TNFα). The levels of gene expression (i.e., a gene expression pattern) can be quantified by Northern blot analysis or RT-PCR, by measuring the amount of protein produced, or by measuring the levels of activity of ApoD or other genes, all by methods well known to those of ordinary skill in the art. In this way, the gene expression pattern can serve as a marker, indicative of the physiological response of the cells to the LXR agonist. Accordingly, this response state may be determined before, and at various points during, treatment of the individual with the LXR agonist.

The present invention also provides a method for monitoring the effectiveness of treatment of a subject with an LXR agonist comprising the steps of (i) obtaining a pre-administration sample from a subject prior to administration of the LXR agonist; (ii) detecting the level of expression of ApoD and/or the level of aggrecanase activity and/or the level of elaboration of pro-inflammatory cytokines in the pre-administration sample; (iii) obtaining one or more post-administration samples from the subject; (iv) detecting the level of expression or activity of ApoD and/or the level of aggrecanase activity and/or the level of elaboration of pro-inflammatory cytokines in the post-administration samples; (v) comparing the level of expression of ApoD and/or the level of aggrecanase activity and/or the level of elaboration of pro-inflammatory cytokines in the pre-administration sample with the ApoD expression and/or aggrecanase activity and/or the level of elaboration of pro-inflammatory cytokines in the post administration sample or samples; and (vi) altering the administration of the LXR agonist to the subject accordingly. For example, increased administration of the LXR agonist may be desirable to increase ApoD expression to higher levels than detected and/or reduce aggrecanase activity to lower levels than detected and/or reduce elaboration of pro-inflammatory cytokines to lower levels than detected, that is, to increase the effectiveness of the LXR agonist. Alternatively, decreased administration of the LXR agonist may be desirable to decrease ApoD expression to lower levels than detected or activity and/or to increase aggrecanase activity to higher levels than detected and/or to increase elaboration of pro-inflammatory cytokines to higher levels than detected, that is, to decrease the effectiveness of the LXR agonist. According to such an embodiment, ApoD expression and/or aggrecanase activity and/or pro-inflammatory cytokine elaboration may be used as an indicator of the effectiveness of an LXR agonist, even in the absence of an observable phenotypic response.

Furthermore, in the treatment of osteoarthritis, compositions containing LXR agonists can be administered exogenously, and it would likely be desirable to achieve certain target levels of LXR agonist in sera, in any desired tissue compartment, and/or in the affected tissue. It would, therefore, be advantageous to be able to monitor the levels of LXR agonist in a patient or in a biological sample including a tissue biopsy sample obtained from a patient and, in some cases, also monitoring the levels of ApoD expression and/or aggrecanase activity and/or pro-inflammatory cytokine elaboration. Accordingly, the present invention also provides methods for detecting the presence of LXR agonist in a sample from a patient.

### V. SCREENING ASSAYS

In one embodiment, expression levels of LXR-responsive genes or activity levels of proteins therefrom can be used to facilitate design and/or identification of compounds that treat osteoarthritis through an LXR-based mechanism. Accordingly, the invention provides methods (also referred to herein as “screening assays”) for identifying modulators, i.e., LXR agonists, that have a stimulatory or inhibitory effect on, for example, ApoD expression and/or aggrecanase activity and/or cytokine elaboration. Compounds thus identified can be used in the treatment of osteoarthritis as described elsewhere herein.

Test compounds can be obtained, for example, using any of the numerous approaches in combinatorial library methods known in the art, including spatially addressable parallel solid phase or solution phase libraries; synthetic library methods requiring deconvolution; the ‘one-bead one-compound’ library method; and synthetic library methods using affinity chromatography selection.

Examples of methods for the synthesis of molecular libraries can be found in, for example: DeWitt S H et al., Proc. Natl. Acad. Sci. U.S.A. 90:6909-13 (1993); Erb E et al., Proc. Natl. Acad. Sci. USA 91:11422-26 (1994); Zuckermann R N et al., J. Med. Chem. 37:2678-85 (1994); Cho C Y et al., Science 261:1303-05 (1993); Carrell et al., Angew. Chem. Int. Ed. Engl. 33:2059 (1994); Carrell et al., Angew. Chem. Int. Ed. Engl. 33:2061 (1994); Gallop M A et al., J. Med. Chem. 37:1233-51 (1994).

Libraries of compounds may be presented in solution (e.g., Houghten R A et al., Biotechniques 13:412-21 (1992)), or on beads (Houghten R A et al., Nature 354:82-84 (1991)), chips (Fodor S A et al., Nature 364:555-56 (1993)), bacteria (U.S. Pat. No. 5,223,409), spores (U.S. Pat. No. 5,223,409), plasmids (Cull M G et al., Proc. Natl. Acad. Sci. USA 89:1865-69 (1992)) or on phage (Scott J K & Smith G P, Science 249:386-90 (1990); Devlin J J et al., Science 249:404-06 (1990); Cwirla S E et al., Proc. Natl. Acad. Sci. 87:6378-82 (1990); Felici F et al., J. Mol. Biol. 222:301-10 (1991); U.S. Pat. No. 5,223,409.).

An exemplary screening assay is a cell-based assay in which a cell that expresses LXR is contacted with a test compound, and the ability of the test compound to modulate ApoD expression and/or aggrecanase activity and/or cytokine elaboration through an LXR-based mechanism. Determining the ability of the test compound to modulate ApoD expression and/or aggrecanase activity and/or cytokine elaboration can be accomplished by monitoring, for example, DNA, mRNA, or protein levels, or by measuring the levels of activity of ApoD, aggrecanase, and/or TNFα, all by methods well known to those of ordinary skill in the art. The cell, for example, can be of mammalian origin, e.g., human.

Novel modulators identified by the above-described screening assays can be used for treatments as described herein.

### EXAMPLES

The present invention is further defined in the following Examples. It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modification of the invention to adapt it to various uses and conditions.

### Example 1

To identify transcripts expressed in either arthritic or normal articular cartilage, tissue samples were obtained from arthritis patients with end-stage knee replacement and nonarthritic amputee individuals. The presence or absence of arthritis was confirmed by histology.

The Human Genome U95Av2 (HG-U95Av2) GeneChip® Array (Affymetrix, Santa Clara, Calif.) was used for expression profiling. The HG-U95Av2 chip contains 25-mer oligonucleotide probes representing ˜12,000 primarily full-length sequences (˜16 probe pairs/sequence) derived from the human genome. For each probe designed to be perfectly complimentary to a target sequence, a partner probe is generated that is identical except for a single base mismatch in its center. These probe pairs allow for signal quantitation and subtraction of nonspecific noise.

RNA was extracted from individual articular cartilage tissue, converted to biotinylated cRNA, and fragmented according to the Affymetrix protocol. The fragmented cRNAs were diluted in 1× MES buffer containing 100 μg/ml herring sperm DNA and 500 μg/ml acetylated BSA and denatured for 5 min at 99° C. followed immediately by 5 min at 45° C. Insoluble material was removed from the hybridization mixtures by a brief centrifugation, and the hybridization mix was added to each array and incubated at 45° C. for 16 hr with continuous rotation at 60 rpm. After incubation, the hybridization mix was removed and the chips were extensively washed with 6× SSPET and stained with SAPE solution as described in the Affymetrix protocol.

The raw florescent intensity value of each transcript was measured at a resolution of 6 mm with a Hewlett-Packard Gene Array Scanner. GeneChip® software 3.2 (Affymetrix), which uses an algorithm to determine whether a gene is “present” or “absent”, as well as the specific hybridization intensity values or “average differences” of each gene on the array, was used to evaluate the fluorescent data. The average difference for each gene was normalized to frequency values by referral to the average differences of 11 control transcripts of known abundance that were spiked into each hybridization mix according to the procedure of Hill A A et al., Science 290:809-12 (2000). The frequency of each gene was calculated and represents a value equal to the total number of individual gene transcripts per 10⁶ total transcripts.

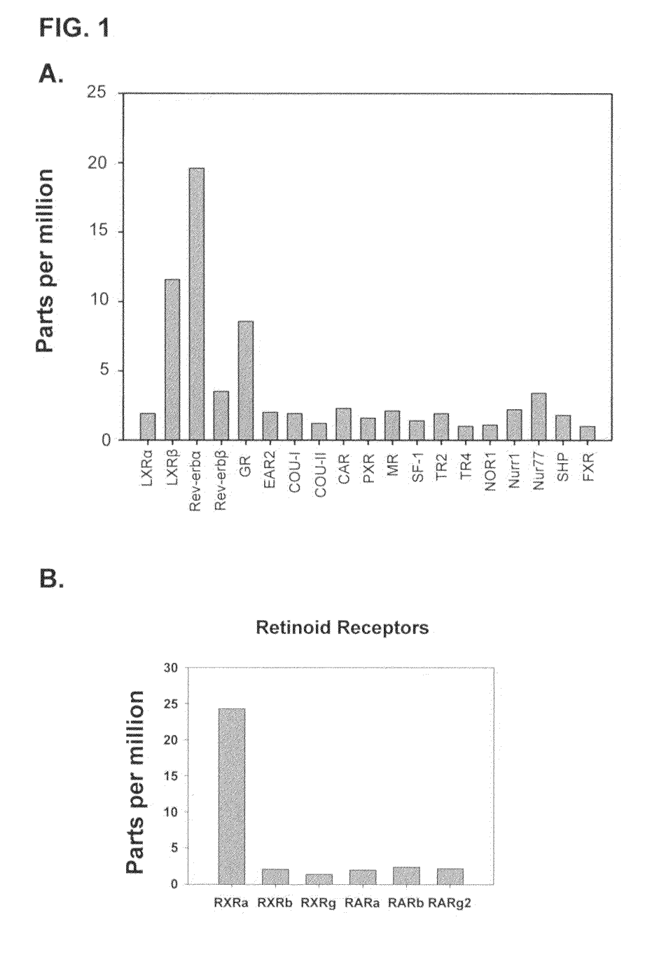

FIG. 1A depicts the mRNA levels in severe osteoarthritic cartilage (expressed as parts per million (ppm)) for 19 different members of the nuclear hormone receptor superfamily (LXRα, LXRβ, Rev-erbα, Rev-erbβ, GR, EAR2, COUP TF-I, COUP TF-II, CAR, PXR, MR, SF-1, TR-2, TR-4, NOR-1, Nurr1, Nur77, SHP, FXR). The lower quantitative limit of detection for these gene chips studies was determined to be approximately 5 ppm. The data shown in FIG. 1 provides evidence that LXRβ, Rev-erbα, and GR appear to be expressed by articular cartilage at the level of sensitivity of the gene chips. In FIG. 1B, the expression levels of the six retinoid receptor family members (Retinoic Acid Receptors (RARs) and Retinoid X Receptors (RXRs)) are shown. These data show that RXRα is expressed in the articular cartilage tissue at levels that are easily detectable. RXRα is a heterodimeric partner of LXR and the biologically active unit of LXR ligand action is LXR-RXR Heterodimer. These data provided an impetus to look at the functional effects of LXR expression in articular cartilage.

### Example 2

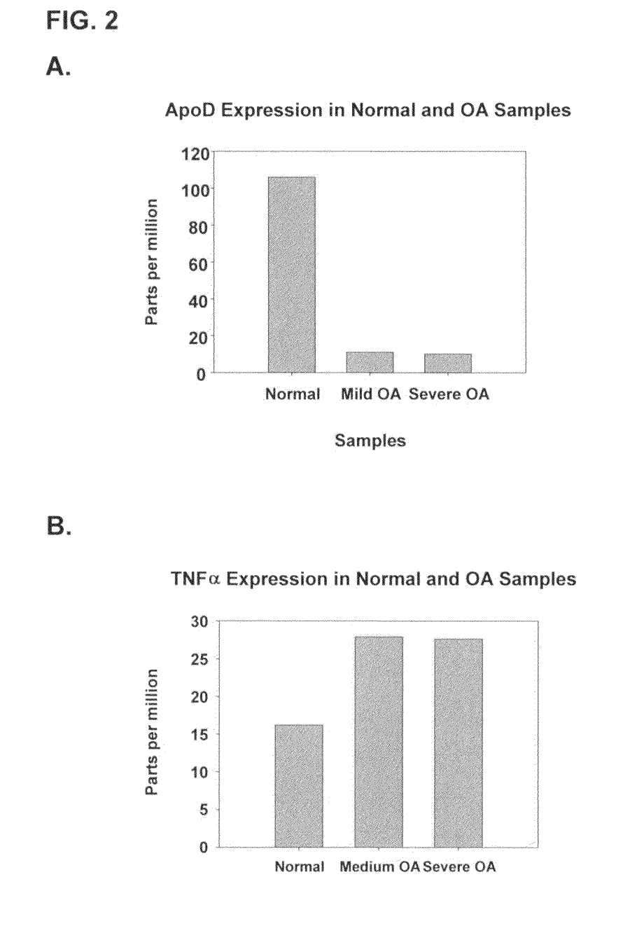

FIG. 2A shows the comparison of ApoD mRNA levels in normal cartilage and cartilage obtained from medium and severe osteoarthritic patients (expressed as parts per million (ppm)). The lower quantitative limit of detection for these gene chips studies was determined to be approximately 5 ppm. The data shown in FIG. 2A provides evidence that the expression of ApoD message is dramatically reduced in mild and severe osteoarthritic cartilage when compared to the normal cartilage. FIG. 2B shows the comparison of TNFα mRNA levels in normal cartilage and cartilage obtained from medium and severe osteoarthritic patients (expressed as parts per million (ppm)). The lower quantitative limit of detection for these gene chips studies was determined to be approximately 5 ppm. The data shown in FIG. 2B provides evidence that the expression of TNFα is significantly induced in mild and severe osteoarthritic cartilage when compared to the normal cartilage.

### Example 3

Fresh cartilage explants (˜20 pieces, a total of ˜200 mg/well) from a human OA donor (#154, from National Disease Research Interchange) were cultured for 10 days in 1 ml of DMEM/F12 containing 1% Nutridoma® (Roche Applied Science, Indianapolis, Ind.). During the 10 days, the explants were exposed to cytokines (1 ng/ml IL1β plus 5 ng/ml Oncostatin M) with or without LXR agonists (2 μM GW3965, a reported LXR agonist, or 2 μM of [4-({3-[3-benzyl-8-(trifluoromethyl)quinolin-4-yl]phenoxy}methyl)phenyl]acetic acid (Formula I shown below), an LXR agonist).

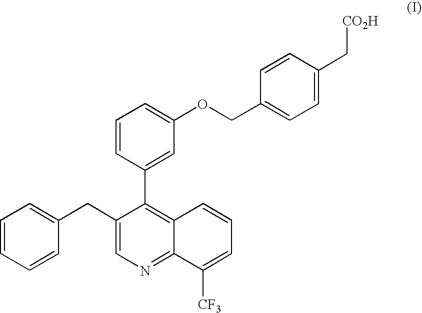

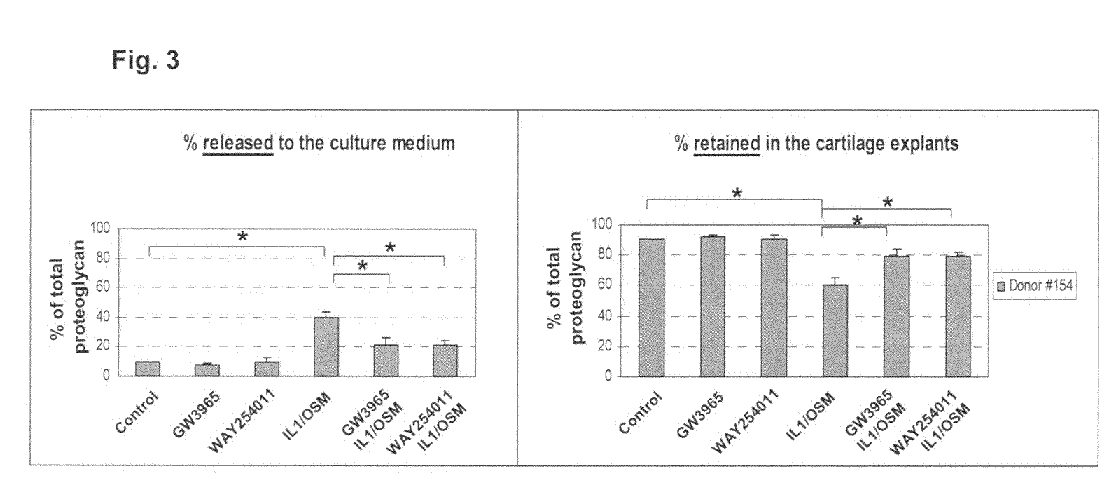

Every 2 days the culture medium was replaced with fresh cytokines and LXR agonists. Accumulative release of proteoglycans was measured in these cultures after using DMMB (dimethylmethylene blue) assay. The explants at the end of the 10-day treatment were then digested with proteinase K and assayed for total proteoglycan content. LXR agonists significantly reduced cytokine-induced release of proteoglycan into the culture medium; consequently, a 10-day treatment of OA cartilage explants with LXR agonist significantly increased total proteoglycan content in the explants (FIG. 3). Since both IL1β and Oncostatin M are present in joints with OA and are believed to play role in OA disease progression, our data suggest that LXR agonist may have a structure-modifying effect in OA cartilage.

### Example 4

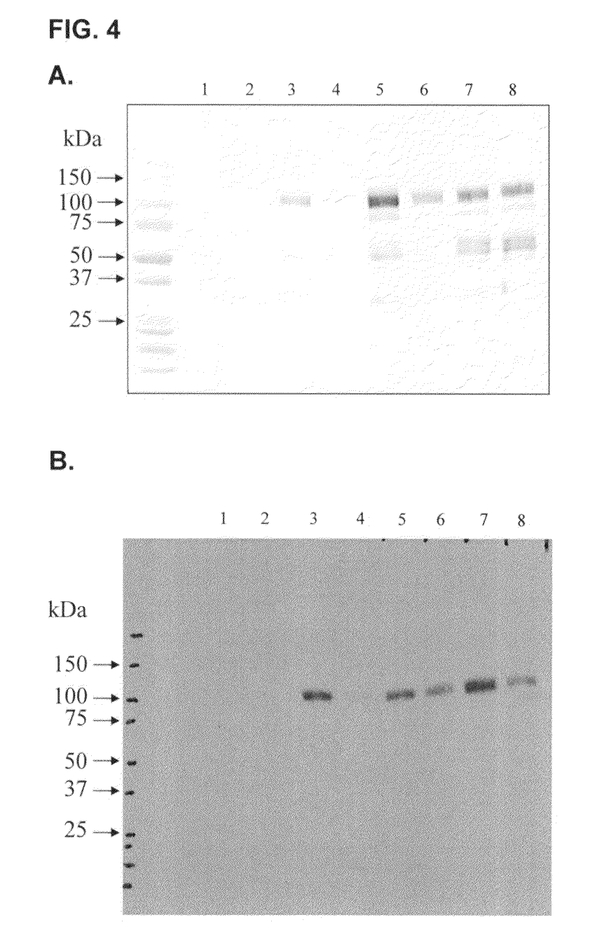

Fresh cartilage from human OA donors was cut into pieces (˜10 mg/piece, ˜2×2×2 mm). The cartilage explants were randomized into 24 well plates (˜250 mg wet weight/well). Three wells of explants were included for each treatment group. The explants were cultured in 1 ml DMEM/F-12 with 10% FBS for 3 days, then the complete medium was replaced with serum-free medium. Twelve hours later, the medium was removed and fresh serum-free medium (1 ml) was added, followed by LXR agonist T0901317 treatment (2 μM). IL1β/Oncostatin M (10 ng/ml each) were added 8 hours later. The explants were then cultured in the presence or absence of LXR agonist T0901317 and IL1β/Oncostatin M for additional 20 hours. 180 μl of pooled culture medium from each treatment group was deglycosylated with chondroitinase ABC, keratanase, keratanase II in the presence of 50 mM EDTA at 37° C. for 3 hrs. The samples were then concentrated and separated in a 4-12% SDS-PAGE gel. Western analysis was performed using either mouse BC3 neoepitope antibody (1:1500), or rabbit anti-AGEG antibody (1:1000) as the primary antibody, and anti-mouse or anti-rabbit IgG antibody conjugated with alkaline peroxidase (1:5000) as the secondary antibody. FIG. 4A shows the result using BC3 antibody, and FIG. 4B shows the result using AGEG antibody. In the experiment using cartilage from donor #259, cytokine treatment induced release of both BC3 and AGEG containing aggrecan fragments into the culture medium. Treatment with T0901317 blocked the induction of BC3 and AEEG release by cytokines. In the experiment using donor #261, BC3- and AEGE-containing aggrecan fragments were released into the culture medium from untreated cartilage explants. T0901317 treatment reduced the amount of these fragments in the culture medium. Release of AGEG-containing fragment from the explants was also induced by cytokine treatment, and it was blocked by T0901317 treatment.

### Example 5

Fresh cartilage explants (˜20 pieces, a total of ˜200 mg/well) from a human OA donor (provided by National Disease Research Interchange) were cultured for 21 days in 1 ml of DMEM/F12 containing 1% Nutridoma® (Roche Applied Science, Indianapolis, Ind.). During the 21 days, the explants were exposed to cytokines (10 ng/ml IL1β plus 10 ng/ml Oncostatin M) with or without LXR agonists (2 μM GW3965 or Formula I). Every 2-3 days the culture medium was replaced with fresh cytokines and LXR agonists. Total amounts of prostaglandin E2 (PGE2) in the culture medium samples collected on day 7, 14, 21 were measured using an EIA assay (Cayman).

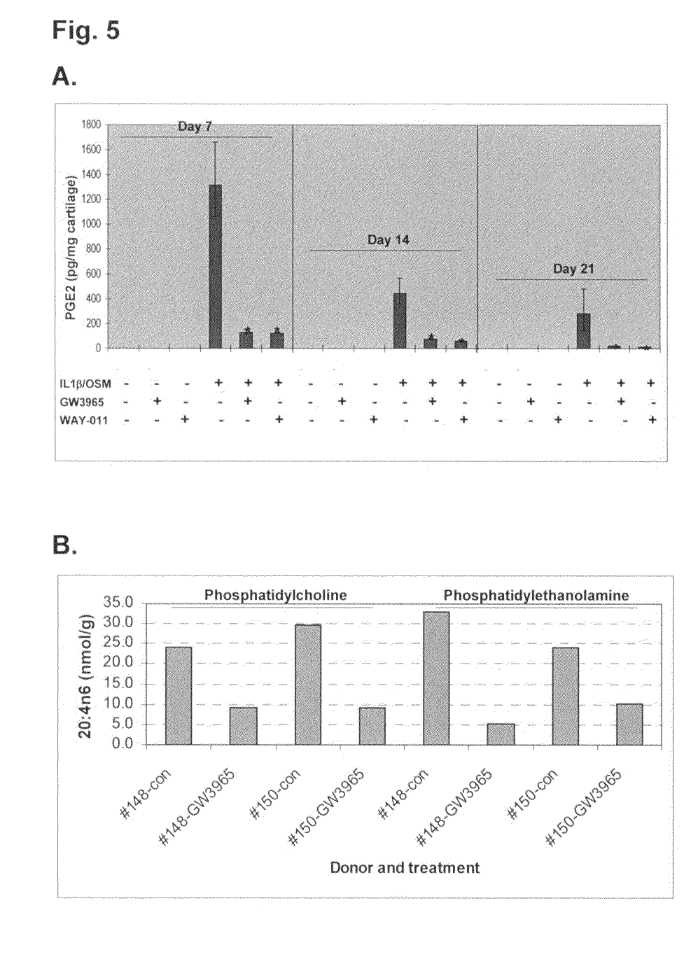

FIG. 5 shows that both LXR agonists strongly inhibit cytokine (IL1β/Oncostatin M)-induced PGE2 synthesis at all 3 time points. Lipid profiling analysis (Lipomics Inc.) results show that the amounts of two forms of membrane phospholipids where most arachidonic acid (AA) is from are reduced by LXR activation, suggesting that the decrease of total PGE2 is mediated at least partly by reduced total AA content in OA cartilage. Expression of enzymes involved in PGE2 synthesis may also be inhibited by LXR activity.

PGE2 is the principal proinflammatory prostanoid found in joints with rheumatoid arthritis (RA) or OA. Increased PGE2 in cartilage may also play a role in inflammation-mediated structural damages that characterize arthritic diseases. More importantly, PGE2 contributes to one of the key features of inflammation, pain hypersensitivity. Therefore, LXR agonists have great potential to be OA therapeutics that will relieve pain by blocking PGE2 production in OA joints, as well as prevent disease-progression by blocking cartilage matrix degradation.

What is claimed is:

1. A method for the treatment of a mammal suffering from osteoarthritis comprising administering to the mammal in need thereof an LXR-responsive gene expression-modulating amount of an LXR agonist.

2. The method of claim 1, wherein the LXR agonist is a natural oxysterol, a synthetic oxysterol, a synthetic nonoxysterol, or a natural nonoxysterol.

3. The method of claim 1, wherein the LXR agonist is 20(S) hydroxycholesterol, 22(R) hydroxycholesterol, 24(S) hydroxycholesterol, 25-hydroxycholesterol, 24(S), 25 epoxycholesterol, 27-hydroxycholesterol, N,N-dimethyl-3β-hydroxycholenamide, N-(2,2,2-trifluoroethyl)-N-{4-[2,2,2-trifluoro-1-hydroxy-1-(trifluoromethyl)ethyl]phenyl}benzene sulfonamide, [3-(3-(2-chloro-trifluoromethylbenzyl-2,2-diphenylethylamino)propoxy)phenylacetic acid], N-methyl-N-[4-(2,2,2-trifluoro-1-hydroxy-1-trifluoromethyl-1-ethyl)-phenyl]-benzenesulfonamide, 4,5-dihydro-1-(3-(3-trifluoromethyl-7-propyl-benzisoxazol-6-yloxy)propyl)-2,6-pyrimidinedione, 3-chloro-4-(3-(7-propyl-3-trifluoromethyl-6-(4,5)-isoxazolyl)propylthio)-phenyl acetic acid, acetyl-podocarpic dimer, paxilline, desmosterol, or stigmasterol.

4. The method of claim 3, wherein the LXR agonist is N-(2,2,2-trifluoroethyl)-N-[4-(2,2,2-trifluoro-1-hydroxy-1-trifluoromethyl-1-ethyl)-phenyl]-benzenesulfonamide.

5. The method of claim 1, wherein treatment with the LXR agonist inhibits cartilage degradation and induces cartilage regeneration.

6. The method of claim 1, wherein the LXR agonist inhibits aggrecanase activity.

7. The method of claim 1, wherein the LXR agonist inhibits elaboration of pro-inflammatory cytokines and/or inflammatory mediators in osteoarthritic joints.

8. The method of claim 7, wherein the inflammatory mediator is prostaglandin E2.

9. The method of claim 1, wherein treatment with the LXR agonist provides pain relief in osteoarthritic joints.

10. The method of claim 1, wherein the LXR-responsive gene is apolipoprotein D.

11. A method of inducing expression of apolipoprotein D in a mammal having osteoarthritic cartilage comprising administering to the mammal in need thereof an effective amount of an LXR agonist.

12. A method of preventing osteoarthritis comprising:
(a) determining a baseline apolipoprotein D expression level in normal cartilage of a subject; and
(b) maintaining baseline apolipoprotein D expression level in cartilage of the subject via treatment with LXR agonist.

13. A method for the treatment of a mammal suffering from osteoarthritis comprising administering to the mammal in need thereof an aggrecanase activity-inhibiting amount of an LXR agonist.

14. A method of inhibiting activity of aggrecanase in a mammal having osteoarthritic cartilage comprising administering to the mammal in need thereof an effective amount of an LXR agonist.

15. A method for the treatment of a mammal suffering from osteoarthritis comprising administering to the mammal in need thereof an effective amount of an LXR agonist to inhibit elaboration of pro-inflammatory cytokines and lipids in osteoarthritic joints.

16. A method for the treatment of a mammal suffering from osteoarthritis comprising administering to the mammal in need thereof an effective amount of an LXR agonist to relieve pain in osteoarthritic joints.

17. The method of claim 16, wherein the LXR agonist inhibits TNFα expression.

18. A method of detecting an osteoarthritic phenotype in a subject comprising:
(a) determining a baseline apolipoprotein D expression level in normal cartilage;
(b) obtaining a cartilage sample from a subject suspected of having osteoarthritis; and
(c) detecting the level of expression of apolipoprotein D in the sample;
wherein a lower amount of apolipoprotein D expression in the sample compared to baseline apolipoprotein D expression is indicative of osteoarthritis.

19. A method of identifying an LXR ligand capable of reducing an osteoarthritic effect in cartilage comprising:
(a) providing a sample containing LXR;
(b) contacting the sample with a test compound; and
(c) determining whether the test compound induces apolipoprotein D expression, inhibits aggrecanase activity, inhibits elaboration of pro-inflammatory cytokines, or a combination thereof.
